(19) United States
(12) Patent Application Publication
(10) Pub. No.: US 2008/0296287 A1
(43) Pub. Date: Dec. 4, 2008

(54) HEATING DEVICE

(76) Inventor: Steven Yue, Taipei (TW)

Correspondence Address: PERMAN & GREEN, 425 POST ROAD, FAIRFIELD, CT, 06824, US

(21) Appl. No.: 11/957,791

(22) Filed: Dec. 17, 2007

(30) Foreign Application Priority Data
May 30, 2007 (CN) 200710108149.1

(51) Int. Cl. H05B 3/54; A61F 7/00

(52) U.S. Cl. 219/549; 126/204

(57) ABSTRACT

A heating device includes an electrically conductive heating wire unit having two opposite end portions and a middle portion extending between the end portions, a phase-change material enclosing the middle portion of the heating wire unit and that is capable of storing heat through phase changing, and an enclosure enclosing the phase-change material for preventing leakage of the phase-change material.

### CROSS-REFERENCE TO RELATED APPLICATION

This application claims priority of Chinese Application No. 200710108149.1, filed on May 30, 2007.

### BACKGROUND OF THE INVENTION

1. Field of the Invention

This invention relates to a heating device, and more particularly, to a heating device including a phase-change material and that can be arranged by bending thereof so as to optimize heat-release distribution.

2. Description of the Related Art

Referring to FIG. 1, a conventional heating device 100, commonly referred to as a “hand warmer,” comprises a pocket 7 that is filled with a phase-change material 70. Before the user can use the hand warmer 100, the user must pre-heat the hand warmer 100 using an external device or means, such as by putting the hand warmer 100 in hot water or by using a microwave oven, so that the phase-change material 70 is heated. As a result, the phase-change material 70 undergoes phase change and therefore, is able to better store heat.

However, the conventional hand warmer 100 can be used only in an indoor setting where such external devices and means are readily available. Further, heat generated from the conventional hand warmer 100 cannot be sustained for a long time since the phase-change material 70 quickly loses its heat energy and returns to its initial state.

### SUMMARY OF THE INVENTION

Therefore, it is an object of the present invention to provide a heating device that is capable of overcoming the above-mentioned drawbacks of the prior art.

According to the present invention, a heating device includes an electrically conductive heating wire unit having two opposite end portions and a middle portion extending between the end portions, a phase-change material enclosing the middle portion of the heating wire unit and that is capable of storing heat through phase changing, and an enclosure enclosing the phase-change material for preventing leakage of the phase-change material.

### BRIEF DESCRIPTION OF THE DRAWINGS

Other features and advantages of the present invention will become apparent in the following detailed description of the preferred embodiments with reference to the accompanying drawings, of which:

FIG. 1 is a perspective view of a conventional heating device;

FIG. 2 is an exploded perspective view of a heating device according to the first preferred embodiment of the present invention;

FIG. 3 is a schematic view of the heating device of the first preferred embodiment, illustrating an example of how the heating device can be arranged in a specific configuration by bending;

FIG. 4 is a fragmentary side view of the first preferred embodiment, illustrating a temperature sensor disposed on an enclosure of the heating device;

FIG. 5 is a fragmentary side view of the first preferred embodiment, illustrating a temperature controller disposed on the enclosure of the heating device;

FIG. 6 is a cross-sectional view of the heating device of the first preferred embodiment, illustrating a wire support mounted therewithin;

FIG. 7 is an exploded perspective view of a modified example of the first preferred embodiment of the heating device in accordance with the present invention;

FIG. 8 is a cross-sectional view of the second preferred embodiment of a heating device in accordance with the present invention; and

FIG. 9 is a schematic view of the second preferred embodiment, illustrating an exemplary layout of the heating device.

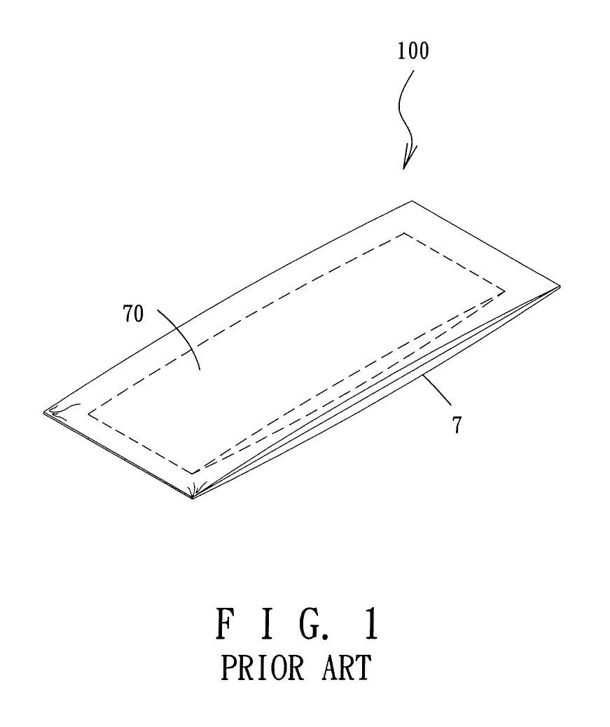

### DETAILED DESCRIPTION OF THE PREFERRED EMBODIMENTS

Before the present invention is described in greater detail with reference to the preferred embodiments, it should be noted herein that like elements are denoted by the same reference numerals throughout the disclosure.

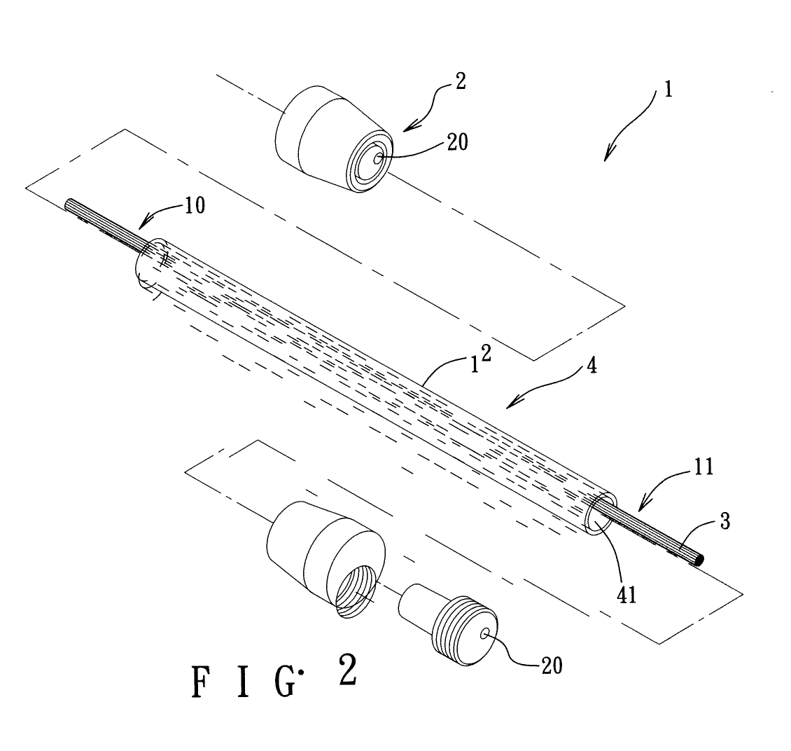

Referring to FIGS. 2 to 6, a heating device 1 according to the first preferred embodiment of the present invention includes an electrically conductive heating wire unit 3 that has two opposite end portions 10, 11 and a middle portion 12 extending between the end portions 10, 11, a phase-change material 6 that encloses the middle portion 12 of the heating wire unit 3 and that is capable of storing heat through phase changing, and an enclosure 4 that encloses the phase-change material 6 for preventing leakage of the phase-change material 6. In the first preferred embodiment, the heating wire unit 3 is an electrothermal wire, which can be a metal- or carbon fiber-type electrothermal wire.

In the first preferred embodiment, the enclosure 4 includes a sleeve 41 and a pair of end caps 2. The phase-change material 6 fills the sleeve 41. The end caps 2 cap two ends of the sleeve 41 and are formed with holes 20 for extension of the end portions 10, 11 of the heating wire unit 3 therethrough, respectively.

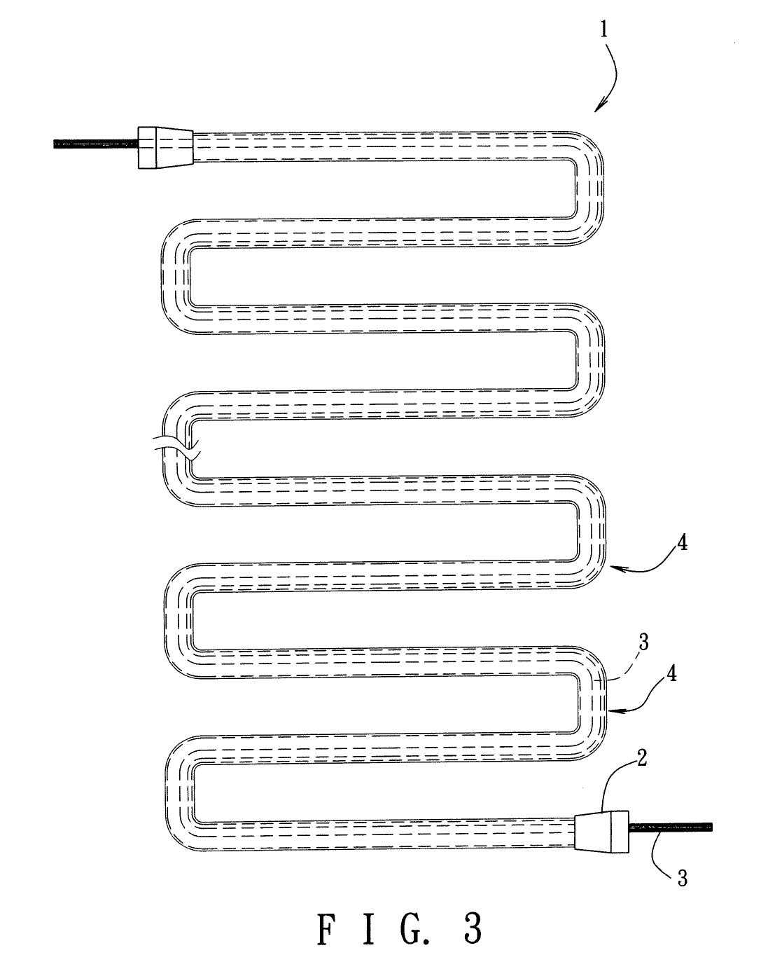

The sleeve 41 of the enclosure 4 is made of a flexible material that allows for reversible reshaping of the sleeve 41. As such, the sleeve 41 is bendable to place the heating device 1 in any arrangement to suit the particular needs of a user. As an example, with reference to FIG. 3, the heating device 1 is arranged in a meandering configuration to cover a relatively large area.

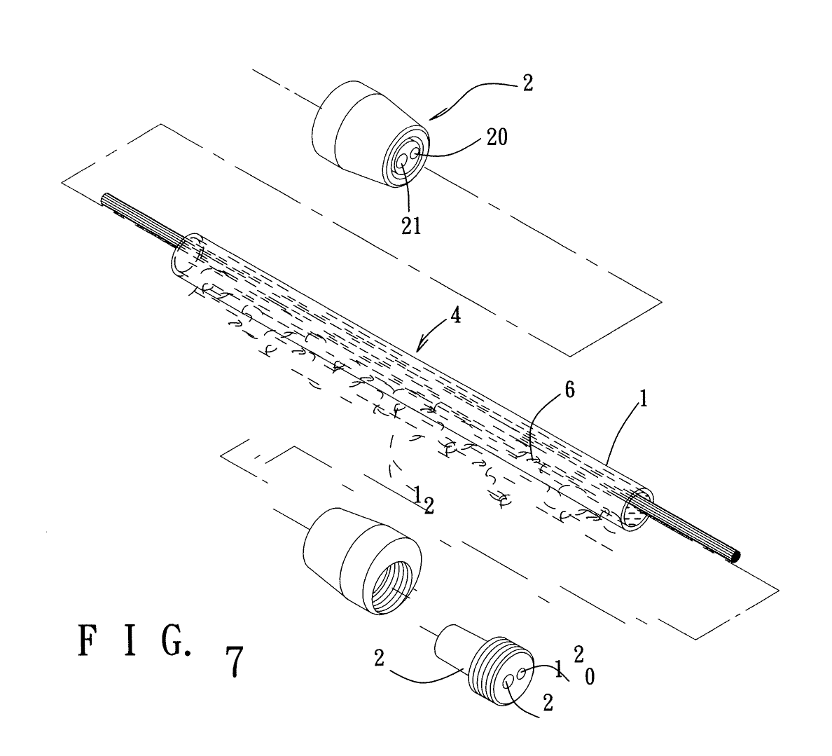

Further, in the first preferred embodiment, with particular reference to FIG. 6, a wire support 5 is disposed in the sleeve 41. The wire support 5 includes a C-shaped ring 50 for holding the heating wire unit 3 therein, and a plurality of protrusions 52 projecting outwardly from the C-shaped ring 50 and that are angularly displaced. A plurality of the wire supports 5 may be disposed in the sleeve 41 to provide full support to the heating wire unit 3.

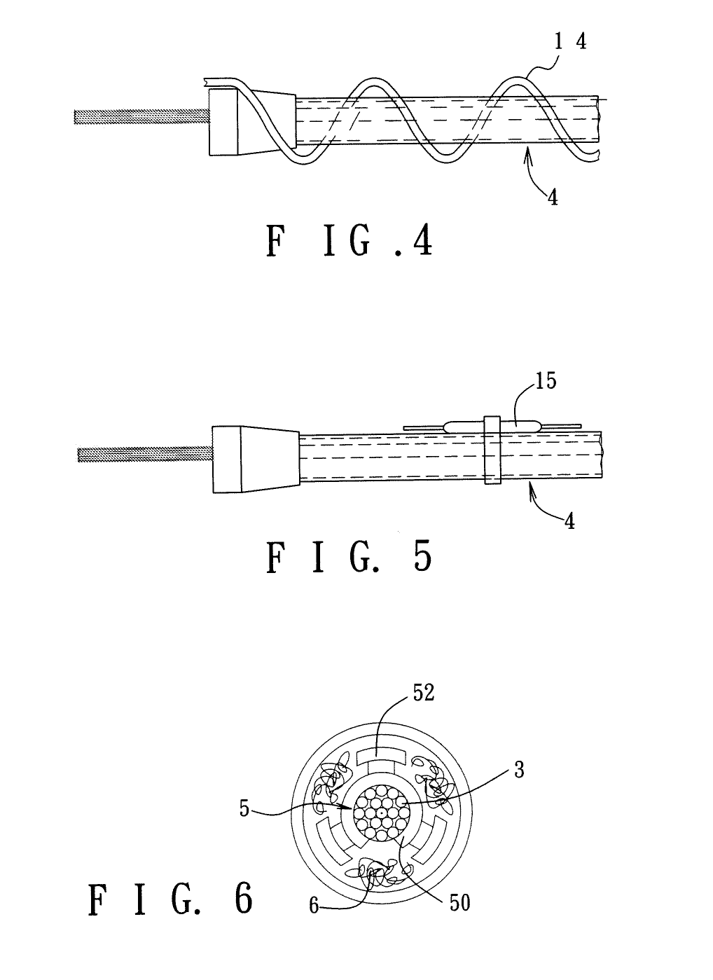

Additionally, with reference to FIGS. 4 and 5, in the first preferred embodiment, a temperature controller 15 and a temperature sensor 14 are separately mounted on the enclosure 4 of the heating device 1. Operations of the temperature controller 15 and the temperature sensor 14 will be described below. In the first preferred embodiment, the end caps 2 are fabricated from the same flexible material as the enclosure 4. Further, the hole 20 in one of the end caps 2 is aligned with the hole 20 in the other of the end caps 2 so as to allow the heating wire unit 3 to extend approximately in a straight line through the sleeve 41 (prior to any bending thereof). The holes 20 are packed with a waterproof material to prevent leakage of the phase-change material 6.

If the heating device 1 is of any considerable length, the heating wire unit 3 may shift downward to thereby contact the inner wall of the sleeve 41 by virtue of gravity acting thereupon. As a result, heat is not uniformly distributed within the enclosure 4. The wire support 5 mounted inside the sleeve 41 prevents the heating wire unit 3 from contacting the inner wall of the sleeve 41. By providing a plurality of the wire supports 5 in the sleeve 41 as described above, the heating wire unit 3 is more fully supported to better prevent the same from contacting the inner wall of the sleeve 41.

In operation, electrical power is supplied to the heating wire unit 3 so that the heating wire unit 3 heats up, i.e., generates heat. The phase-change material 6 absorbs the heat and therefore, undergoes a change in phase so as to better store the heat. This stored heat is then released from the phase-change material 6. Because of the use of the phase-change material 6 in the heating device 1, only a small amount of the electrical power is required by the heating device 1.

It is to be noted that in some embodiments, the phase-change material 6 may be omitted from the configuration of the heating device 1. In this case, the heating device 1 is still operable to generate, store, and emit heat, albeit in a manner less efficiently than when the phase-change material 6 is filled in the enclosure 4.

Referring again to FIG. 4, the temperature sensor 14 is mounted on the enclosure 4 as described above, and is used to monitor the temperature of the heating device 1. Further, as shown in FIG. 5, the temperature controller 15 is mounted on the enclosure 4 and is coupled to the temperature sensor 14. The temperature controller 15 operates in conjunction with the temperature sensor 14 to control the temperature of the heating device 1. Once the temperature of the heating device 1 reaches a predetermined level, the temperature controller 15 will control the supply of the electrical power to the heating wire unit 3 such that the predetermined temperature level is maintained.

FIG. 7 illustrates a modified example of the first preferred embodiment of the heating device 1 according to the present invention. In this modified example of the first preferred embodiment, each of the end caps 2 is further formed with a second hole 21 through which the phase-change material 6 may be filled into the enclosure 4. After the phase-change material 6 fills the enclosure 4, the hole 21 is packed with waterproof material.

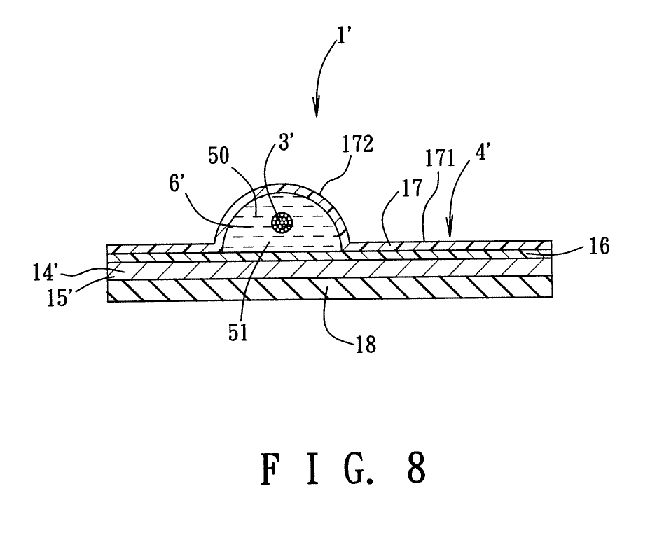

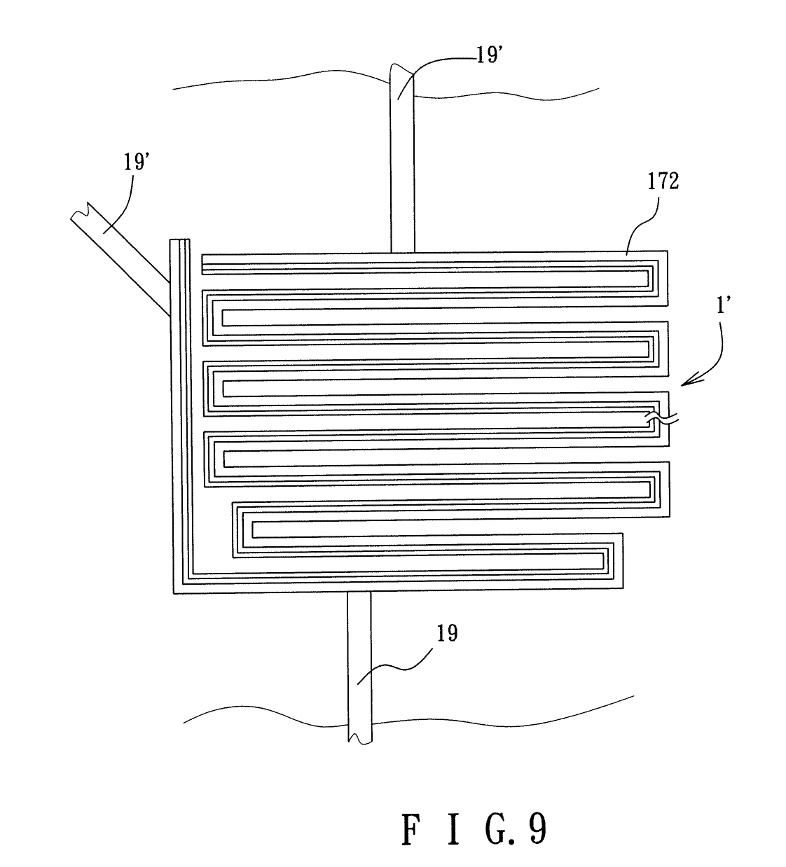

FIG. 8 illustrates the second preferred embodiment of the heating device 1′ of the present invention. In the second preferred embodiment, the enclosure 4′ includes a corrugated layer 17 having a planar portion 171 and a protruding portion 172 protruding from the planar portion 171. The protruding portion 172 defines a groove 50 that has an open end 51. The enclosure 4′ further includes a planar layer 16 that is attached to the corrugated layer 17 so as to cover the open end 51 of the groove 50. The phase-change material 6′ fills the groove 50.

A protective layer 18 is attached to the planar layer 16, and the temperature sensor 14′ and the temperature controller 15′ are sandwiched between the protective layer 18 and the planar layer 16.

In this embodiment, the heating device 1′ can be provided on a piece of clothing (not shown) of the user, such as a jacket. When the heating device 1′ is used for such an application, the protective layer 18 is exposed to the environment, while the heating wire unit 3′ is proximate to the user's body. Because of this arrangement, the protective layer 18 can insulate the heating device 1′ from the cold environment while the heating wire unit 3′ generates heat towards the user's body. As in the case of the first preferred embodiment, the temperature sensor 14′ and the temperature controller 15′ of the second preferred embodiment may operate in combination to control the temperature setting of the heating device 1′.

Reference is now made to FIG. 9, which illustrates an exemplary layout of the heating device 1′ in accordance with the second preferred embodiment. This exemplary layout of the heating device 1′ is realized by forming the groove 50 in a meandering shape. The phase-change material 6′ is supplied to fill the groove 50 through an inlet tube 19, and at least one outlet tube 19′ may be provided on the heating device 1′ so as to discharge air bubbles.

It is to be noted that if any one of the temperature sensor 14′, the temperature controller 15′, the protective layer 18, or the phase-change material 6′ is not included as part of the second preferred embodiment, the heating device 1′ will nevertheless remain operable.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A heating device comprising:
an electrically conductive heating wire unit having two opposite end portions and a middle portion extending between said end portions;
a phase-change material enclosing said middle portion of said heating wire unit and capable of storing heat through phase changing; and
an enclosure enclosing said phase-change material for preventing leakage of said phase-change material.

2. The heating device as claimed in claim 1, wherein said enclosure includes a sleeve and a pair of end caps, said phase-change material filling said sleeve, said end caps capping two ends of said sleeve and being formed with holes for extension of said end portions of said heating wire unit therethrough, respectively.

3. The heating device as claimed in claim 2, further comprising a wire support disposed in said sleeve and including a C-shaped ring for holding said heating wire unit therein, and a plurality of protrusions projecting outwardly from said C-shaped ring and being angularly displaced.

4. The heating device as claimed in claim 2, wherein said sleeve is bendable so as to allow for reversible reshaping of said sleeve.

5. The heating device as claimed in claim 1, wherein said enclosure includes a corrugated layer having a planar portion and a protruding portion protruding from said planar portion and defining a groove that has an open end, said enclosure further including a planar layer attached to said corrugated layer to cover said open end of said groove, said phase-change material filling said groove.

6. The heating device as claimed in claim 5, further comprising a protective layer attached to said planar layer, and a temperature sensor and a temperature controller sandwiched between said protective layer and said planar layer.

7. The heating device as claimed in claim 5, wherein said groove is meandering in shape.

8. The heating device as claimed in claim 1, further comprising a temperature controller and a temperature sensor, each mounted on said enclosure of said heating device.

9. A heating device comprising:
an electrically conductive heating wire unit having two opposite end portions and a middle portion extending between said end portions; and
an enclosure including a sleeve and a pair of end caps, said end caps capping two ends of said sleeve and being formed with holes for extension of said end portions of said heating wire unit therethrough, respectively.

10. The heating device as claimed in claim 9, wherein said sleeve is bendable so as to allow for reversible reshaping of said sleeve.
